(19) United States
(12) Patent Application Publication
(10) Pub. No.: US 2011/0082209 A1
(43) Pub. Date: Apr. 7, 2011

(54) Novel Salts of (4--2-Methylphenyl)Carbonyl]Amino}-3-Methylphenyl)Acetic Acid

(76) Inventors: Trevor Raymond Keel, Hertfordshire (GB); Sarah Vallance, Hertfordshire (GB)

(21) Appl. No.: 12/997,062

(22) PCT Filed: Jun. 8, 2009
(86) PCT No.: PCT/EP09/57007

§ 371 Date: Dec. 9, 2010

(30) Foreign Application Priority Data
Jun. 10, 2008 (GB) 0810615.5

(51) Int. Cl. A61K 31/196; C07C 235/56; A61P 19/08; A61P 29/02; A61P 35/00; A61P 9/00; A61P 25/04; A61P 25/06; A61P 1/00

(52) U.S. Cl. 514/563; 562/455

(57) ABSTRACT

A series of novel salt forms of (4-{[(5-{[(3-chlorophenyl)methyl]oxy}-2-methylphenyl)carbonyl]amino}-3-methylphenyl)acetic acid, pharmaceutical compositions comprising such compounds and the use of such compounds in medicine.

This invention relates to a novel pharmaceutical, to a process for the preparation of the pharmaceutical and to the use of the pharmaceutical in medicine.

International Patent Application Number PCT/EP2007/063796 (WO2008/071736) discloses certain benzamide derivatives as EP4 receptor agonists including (4-{[(5-{[(3-chlorophenyl)methyl]oxy}-2-methylphenyl)carbonyl]amino}-3-methylphenyl)acetic acid.

A series of novel salt forms of (4-{[(5-{[(3-chlorophenyl)methyl]oxy}-2-methylphenyl)carbonyl]amino}-3-methylphenyl)acetic acid have been identified which may have useful pharmaceutical properties and in particular they are indicated to be useful for the treatment and/or prophylaxis of diseases and disorders including, but not limited to pain and Bone Disorders as hereinbelow defined.

Thus, according to a first aspect of the invention there is provided a compound which is (4-{[(5-{[(3-chlorophenyl)methyl]oxy}-2-methylphenyl)carbonyl]amino}-3-methylphenyl)acetate sodium salt.

According to a second aspect of the invention there is provided a compound which is (4-{[(5-{[(3-chlorophenyl)methyl]oxy}-2-methylphenyl)carbonyl]amino}-3-methylphenyl)acetate potassium salt.

According to a third aspect of the invention there is provided a compound which is (4-{[(5-{[(3-chlorophenyl)methyl]oxy}-2-methylphenyl)carbonyl]amino}-3-methylphenyl)acetate methyl-D-glucamine salt.

According to a fourth aspect of the invention there is provided a compound which is (4-{[(5-{[(3-chlorophenyl)methyl]oxy}-2-methylphenyl)carbonyl]amino}-3-methylphenyl)acetate tris(hydroxymethyl)-aminomethane salt.

It will be appreciated that references herein to “compounds” or “salt forms” refer to the sodium, potassium, methyl-D-glucamine and tris(hydroxymethyl)-aminomethane salts of (4-{[(5-{[(3-chlorophenyl)methyl]oxy}-2-methylphenyl)carbonyl]amino}-3-methylphenyl)acetic acid.

(4-{[(5-{[(3-chlorophenyl)methyl]oxy}-2-methylphenyl)carbonyl]amino}-3-methylphenyl)acetic acid may be prepared according to known procedures, such as those disclosed in International Patent Application Number PCT/EP2007/063796 (WO2008/071736). The disclosure of PCT/EP2007/063796 is incorporated herein by reference.

When some of the compounds of this invention are allowed to crystallise or are recrystallised from organic solvents, solvent of crystallisation may be present in the crystalline product. This invention includes within its scope such solvates of the salt forms of the invention. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the salt forms of the invention.

As mentioned above the compounds of the invention have useful therapeutic properties. More particularly, the compounds of the present invention are believed to be of potential use in the treatment or prophylaxis of diseases or disorders where an EP4 receptor agonist is required such as pain, for example, chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The compounds may be particularly useful in the treatment of neuropathic pain and symptoms associated therewith. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. Symptoms of neuropathic pain include spontaneous shooting and lancinating pain, or ongoing, burning pain. In addition, there is included pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, COPD; gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease, diarrhea, constipation); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, polymyositis, tendinitis, bursitis, and Sjogren's syndrome.

The compounds may also be useful in the treatment of immunological diseases such as autoimmune diseases, immunological deficiency diseases or organ transplantation. The compounds may also be effective in increasing the latency of HIV infection.

The compounds may also be useful in the treatment of diseases of excessive or unwanted platelet activation such as intermittent claudication, unstable angina, stroke, and acute coronary syndrome (e.g. occlusive vascular diseases).

The compounds may also be useful as a drug with diuretic action, or may be useful to treat overactive bladder syndrome.

The compounds may also be useful in the treatment of impotence or erectile dysfunction.

The compounds of formula (I) may also be useful in the treatment of various Bone Disorders as hereinbelow defined, which includes the treatment of bone fractures, bone injury or bone defects.

For example, the compounds of the invention may be useful in enhancement of bone formation i.e. osteogenesis, such as increasing bone mass, bone volume, osteoblast number or osteoblast survival.

The compounds of formula (I) may therefore be useful in the treatment of bone disease, including genetic disorders, that are characterised by abnormal bone metabolism or resorption such as osteoporosis (especially postmenopausal osteoporosis, glucocorticoid induced osteoporosis, hyperthyroidism-induced osteoporosis, immobilisation-induced osteoporosis, heparin-induced osteoporosis and immunosuppressive-induced osteoporosis as well as long term complications of osteoporosis such as curvature of the spine, loss of height and prosthetic surgery), abnormally increased bone turnover, hyper-calcemia (including humoral hypercalcemia), hyperparathyroidism, Paget's bone diseases, osteolysis (including periprosthetic osteolysis), hypercalcemia of malignancy with or without bone metastases, hypercalcemia of fracture healing, rheumatoid arthritis, osteoarthritis (including disease modifying in osteoarthristis such as cartilage/bone repair), ostealgia, osteopenia, calculosis, lithiasis (especially urolithiasis), gout and ankylosing spondylitis, tendonitis, bursitis, malignant bone tumour e.g. osteosarcoma, osteogenesis imperfecta, metastatic bone disease, alveolar bone loss, post-osteomy and childhood idiopathic bone loss.

The compounds of formula (I) may also be useful in bone remodelling and/or promoting bone generation and/or promoting fracture healing. For example, the compounds of the present invention may be useful in fracture healing e.g. long bone fractures and fractures of other bones. The compounds of the present invention may also be useful in healing fractures of the head, face and neck caused e.g. by injury. The compounds of the present invention may also be useful in bone grafting including replacing bone graft surgery entirely, enhancing the rate of successful bone grafts, bone healing following facial reconstruction, maxillary reconstruction, mandibular reconstruction, craniofacial reconstruction e.g. of craniofacial defects such as orofacial defects at birth (including orofacial clefts such as cleft palate), prosthetic ingrowth, vertebral synostosis, long bone extension, spinal fusion, and sternotomy. The compounds of the invention may also be useful in treating bone defects that might evolve around defects that occur during war.

The compounds of the invention may also be useful in periodontal indications such as periodontal disease (periodontitis), tooth loss, and peridontal augmentation e.g. in preparation for tooth implants.

The compounds of the present invention may also be useful in facilitating joint fusion, facilitating tendon and ligament repair, reducing the occurrence of secondary fracture, treating avascular necrosis, facilitating cartilage repair, facilitating bone healing after limb transplantation and repairing damage caused by metastatic bone disease.

The compounds may also be useful for attenuating the hemodynamic side effects of NSAIDs and COX-2 inhibitors.

The compounds may also be useful in the treatment of cardiovascular diseases such as hypertension or myocardial ischemia; functional or organic venous insufficiency; varicose therapy; haemorrhoids; and shock states associated with a marked drop in arterial pressure (e.g. septic shock).

The compounds may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds may also be useful in the treatment of neurological disorders and may be useful as neuroprotecting agents. The compounds may also be useful in the treatment of neurodegeneration following stroke, cardiac arrest, pulmonary bypass, traumatic brain injury, spinal cord injury or the like.

The compounds may also be useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds may also be useful in the treatment of kidney dysfunction (nephritis, particularly mesangial proliferative glomerulonephritis, nephritic syndrome), liver dysfunction (hepatitis, cirrhosis) and gastrointestinal dysfunction (diarrhea).

It is to be understood that as used herein any reference to treatment includes both treatment of established symptoms and prophylactic treatment.

According to a further embodiment the invention, there is provided a salt form of (4-{[(5-{[(3-chlorophenyl)methyl]oxy}-2-methylphenyl)carbonyl]amino}-3-methylphenyl)acetic acid as defined hereinbefore for use in human or veterinary medicine.

According to another embodiment of the invention, there is provided a salt form of (4-{[(5-{[(3-chlorophenyl)methyl]oxy}-2-methylphenyl)carbonyl]amino}-3-methylphenyl)acetic acid as defined hereinbefore for use in the treatment of a condition which is mediated by the action, or loss of action, of PGE₂ at EP₄ receptors. According to another embodiment of the invention, there is provided a salt form of (4-{[(5-{[(3-chlorophenyl)methyl]oxy}-2-methylphenyl)carbonyl]amino}-3-methylphenyl)acetic acid as defined hereinbefore for use in the treatment of a Bone Disorder.

According to a further embodiment of the invention, there is provided a method of treating a human or animal subject suffering from a condition which is mediated by the action, or by loss of action, of PGE₂ at EP₄ receptors which comprises administering to said subject an effective amount of a salt form of (4-{[(5-{[(3-chlorophenyl)methyl]oxy}-2-methylphenyl)carbonyl]amino}-3-methylphenyl)acetic acid as defined hereinbefore. According to a further embodiment of the invention, there is provided a method of treating a human or animal subject suffering from a Bone Disorder which comprises administering to said subject an effective amount of a salt form of (4-{[(5-{[(3-chlorophenyl)methyl]oxy}-2-methylphenyl)carbonyl]amino}-3-methylphenyl)acetic acid as defined hereinbefore.

According to a further embodiment of the invention there is provided a method of treating a human or animal subject suffering from a pain, or an inflammatory, immunological, neurodegenerative or renal disorder, which method comprises administering to said subject an effective amount of a salt form of (4-{[(5-{[(3-chlorophenyl)methyl]oxy}-2-methylphenyl)carbonyl]amino}-3-methylphenyl)acetic acid as defined hereinbefore.

According to another embodiment of the invention, there is provided the use of a salt form of (4-{[(5-{[(3-chlorophenyl)methyl]oxy}-2-methylphenyl)carbonyl]amino}-3-methylphenyl)acetic acid as defined hereinbefore for the manufacture of a medicament for the treatment of a condition which is mediated by the action, or loss of action, of PGE₂ at EP₄ receptors. According to another embodiment of the invention, there is provided the use of a salt form of (4-{[(5-{[(3-chlorophenyl)methyl]oxy}-2-methylphenyl)carbonyl]amino}-3-methylphenyl)acetic acid as defined hereinbefore for the manufacture of a medicament for the treatment of a Bone Disorder.

According to another embodiment of the invention there is provided the use of a salt form of (4-{[(5-{[(3-chlorophenyl)methyl]oxy}-2-methylphenyl)carbonyl]amino}-3-methylphenyl)acetic acid as defined hereinbefore for the manufacture of a medicament for the treatment or prevention of a condition such as a pain, or an inflammatory, immunological, neurodegenerative or renal disorder.

The compounds of the invention are conveniently administered in the form of pharmaceutical compositions. Such compositions may conveniently be presented for use in conventional manner in admixture with one or more physiologically acceptable carriers or diluents.

Thus, in another aspect of the invention, there is provided a pharmaceutical composition comprising a salt form of (4-{[(5-{[(3-chlorophenyl)methyl]oxy}-2-methylphenyl)carbonyl]amino}-3-methylphenyl)acetic acid as defined hereinbefore adapted for use in human or veterinary medicine.

While it is possible for the compounds to be administered as the raw chemical, it is preferable to present them as a pharmaceutical formulation. The formulations of the present invention comprise the salt forms of (4-{[(5-{[(3-chlorophenyl)methyl]oxy}-2-methylphenyl)carbonyl]amino}-3-methylphenyl)acetic acid as defined hereinbefore together with one or more acceptable carriers or diluents therefor and optionally other therapeutic ingredients. The carrier(s) must be “acceptable” in the sense of being compatible with the other ingredients of the formulation and not deleterious to the recipient thereof. Thus, in one embodiment the invention provides a pharmaceutical composition comprising a salt form of (4-{[(5-{[(3-chlorophenyl)methyl]oxy}-2-methylphenyl)carbonyl]amino}-3-methylphenyl)acetic acid as defined hereinbefore and a pharmaceutically acceptable carrier or diluent therefor.

Administration of the compounds of this invention can be via any method which delivers a compound of this invention systemically and/or locally. The formulations include those suitable for oral, parenteral (including subcutaneous e.g. by injection or by depot, intradermal, intrathecal, intracapsular, intraspinal, intrasternal, intra-articular, intramuscular e.g. by depot, intravenous and intranasal), rectal and topical (including dermal, buccal and sublingual) administration although the most suitable route may depend upon for example the condition and disorder of the recipient. The formulations may conveniently be presented in unit dosage form and may be prepared by any of the methods well known in the art of pharmacy (see for example methods disclosed in ‘Remington—The Science and Practice of Pharmacy’, 21^{st} Edition, Lippincott, Williams & Wilkins, USA, 2005 and references therein). All methods include the step of bringing into association the active ingredient with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

The compounds may also be formulated as depot preparations. Such long acting formulations may be administered by implantation (for example subcutaneously or intramuscularly) or by injection (for example intramuscular or intra-articular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Local application (e.g., to the site of bone fracture, intra-articular) may be achieved by injection of the compound in a suitable solvent or, in cases of open surgery, by local application thereto of such compounds in a suitable carrier. Alternatively, local application may be achieved by applying a solution or dispersion of the compound in a suitable carrier or diluent onto the surface of, or incorporating it into, solid or semi-solid implants conventionally used in orthopedic surgery.

The present invention can also be administered using an injectable, flowable composition that provides sustained release at the local site of the injection by forming a biodegradable solid or gel depot, matrix or implant.

In addition to the ingredients particularly mentioned above, the formulations may include other agents conventional in the art having regard to the type of formulation in question, for example those suitable for oral administration may include flavouring agents.

The compounds may be used in combination with other therapeutic agents, for example COX-2 inhibitors, such as celecoxib, rofecoxib, valdecoxib or parecoxib; 5-lipoxygenase inhibitors; analgesics such as paracetamol; NSAID's, such as diclofenac, indomethacin, nabumetone, naproxen or ibuprofen; leukotriene receptor antagonists; DMARD's such as methotrexate; sodium channel blockers, such as lamotrigine; N-type calcium channel antagonists; NMDA receptor modulators, such as glycine receptor antagonists; gabapentin, pregabalin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₁ receptor ligands; EP₂ receptor ligands; EP₃ receptor ligands; EP, antagonists; EP₂ antagonists and EP₃ antagonists; cannabanoid receptor agonists; VR1 antagonists. When the compounds are used in combination with other therapeutic agents, the compounds may be administered either sequentially or simultaneously by any convenient route.

The compounds of the invention may also be used in combination with known agents useful for treating or preventing the bone disorders described above. The present invention therefore includes combinations of the presently disclosed compounds with other agents including the following: Progestins (such as algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, fluorogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol), polyphosphonates (such as geminal diphosphonates (also referred to as bis-phosphonates), tiludronate disodium, ibandronic acid, alendronate and zoledronic acid), bisphosphonate(s), estrogen agonists/antagonists (such as droloxifene, tamoxifen, 4-hydroxy tamoxifen, raloxifene, toremifene, centchroman, levormeloxifene and idoxifene), estrogen, an estrogen receptor modulator, estrogen/progestin combinations, an androgen receptor modulator, Premarin®, estrone, estriol or 17α- or 17β-ethynyl estradiol, other anti bone-resorptive agents, other bone anabolic agents, also referred to as bone mass augmenting agents, a prostaglandin, or prostaglandin agonist/antagonist, IGF-1, sodium fluoride, parathyroid hormone (PTH), active fragments of parathyroid hormone, growth hormone or growth hormone secretagogues, a cathepsin K inhibitor, an inhibitor of osteoclast proton ATPase, an inhibitor of HMG-CoA reuctase, an integrin receptor antagonist, an osteoblast anabolic agent such as PTH, calcitonin, Vitamin D or a synthetic Vitamin D analogue, and the pharmaceutically acceptable salts and mixtures thereof.

The invention thus provides, in a further embodiment, a combination comprising a salt form of (4-{[(5-{[(3-chlorophenyl)methyl]oxy}-2-methylphenyl)carbonyl]amino}-3-methylphenyl)acetic acid as defined hereinbefore together with a further therapeutic agent or agents. In a further embodiment of the invention there is provided a combination comprising a salt form of (4-{[(5-{[(3-chlorophenyl)methyl]oxy}-2-methylphenyl)carbonyl]amino}-3-methylphenyl)acetic acid as defined hereinbefore and paracetamol.

The combinations referred to above may conveniently be presented for use in the form of a pharmaceutical formulation and thus pharmaceutical formulations comprising a combination as defined above together with a pharmaceutically acceptable carrier or diluent comprise a further aspect of the invention. The individual components of such combinations may be administered either sequentially or simultaneously in separate or combined pharmaceutical formulations.

When a compound of the invention is used in combination with a second therapeutic agent active against the same disease, the dose of each compound may differ from that when the compound is used alone. Appropriate doses will be readily appreciated by those skilled in the art.

In one embodiment of the invention there is provided a method of treating a human or animal subject suffering from a condition which is mediated by the action, or by loss of action, of PGE₂ at EP₄ receptors which comprises administering to said subject an effective amount of a salt form of (4-{[(5-{[(3-chlorophenyl)methyl]oxy}-2-methylphenyl)carbonyl]amino}-3-methylphenyl)acetic acid as defined hereinbefore and paracetamol. In one embodiment of the invention there is provided a method of treating a human or animal subject suffering from a Bone Disorder which comprises administering to said subject an effective amount of a salt form of (4-{[(5-{[(3-chlorophenyl)methyl]oxy}-2-methylphenyl)carbonyl]amino}-3-methylphenyl)acetic acid as defined hereinbefore and paracetamol.

A proposed daily dosage of the compound for the treatment of man is from 0.001 to 30 mg/kg body weight per day and more particularly 0.1 to 3 mg/kg body weight per day, calculated as the free acid, which may be administered as a single or divided dose, for example one to four times per day. The dose range for adult human beings is generally from 0.1 to 1000 mg/day, such as from 10 to 800 mg/day, preferably 10 to 200 mg/day, calculated as the free acid.

A suitable daily dosage of paracetamol is up to 4000 mg per day. Suitable unit doses include 200, 400, 500 and 1000 mg, one, two, three or four times per day.

The precise amount of the compound administered to a host, particularly a human patient, will be the responsibility of the attendant physician. However, the dose employed will depend on a number of factors including the age and sex of the patient, the precise condition being treated and its severity, the route of administration, and any possible combination therapy that may be being undertaken.

The following examples illustrate the invention but do not limit it in any way.

### Analytical Procedures

For LC/MS data the 5 minute method is used unless stated otherwise.

### LC/MS—5 Minute Method:

### Hardware

Agilent 1100 Gradient Pump
Agilent 1100 Autosampler
Agilent 1100 DAD Detector
Agilent 1100 Degasser
Agilent 1100 Oven
Agilent 1100 Controller
Waters ZQ Mass Spectrometer or Waters ZMD Mass Spectrometer
Sedere Sedex 75, Sedere Sedex 85 or Polymer Labs PL-ELS-2100

### Software

Waters MassLynx version 4.0 SP2

### Column

The column used is a Waters Atlantis, the dimensions of which are 4.6 mm×50 mm. The stationary phase particle size is 3 um.

### Solvents

A: Aqueous solvent=Water+0.05% Formic Acid
B: Organic solvent=Acetonitrile+0.05% Formic Acid

### Method

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/mins.
The injection volume for the generic method is 5 ul
The column temperature is 30 deg
The UV detection range is from 220 to 330 nm

All retention times are measured in minutes.

### LC/MS—2 Minute Method:

### Hardware

### Waters Acquity Binary Solvent Manager

### Waters Acquity Sample Manager

### Waters Acquity PDA

### Waters ZQ Mass Spectrometer

### Sedere Sedex 75, Sedere Sedex 85 or Polymer Labs PL-ELS-2100

### Software

Waters MassLynx version 4.1

### Column

Acquity HPLC BEH C₁₈ 1.7 μm 2.1 mm×50 mm
Column oven set to 40 degrees centigrade

### Solvents

A: Aqueous solvent=Water 0.1% Formic Acid+10 mM Ammonium Acetate
B: Organic solvent=MeCN:Water 95:5+0.05% Formic Acid
Weak wash Solvent=MeOH:Water 50:50

### Strong Wash Solvent=MeOH

### Instrument Settings

Injection volume: 0.5 μl
Injection technique: Partial loop overfill

### Weak Wash: 500 μl

### Strong Wash: 500 μl

UV detection: 220 to 330 nm
UV sampling rate: 40 points per second
MS scan range: 100 to 1000 amu
MS scanning rate: 0.2 second scan with a 0.1 second inter scan delay
MS scan function: Electrospray with pos neg switching
Cycle time: 2 minutes and 30 seconds

### Gradient

| Time | Flow ml/min | % A | % B | Curve |
|---|---|---|---|---|
| 0 | 1 | 97 | 3 | 6 |
| 0.1 | 1 | 97 | 3 | 6 |
| 1.4 | 1 | 0 | 100 | 6 |
| 1.9 | 1 | 0 | 100 | 6 |
| 2 | 1 | 97 | 3 | 6 |

### NMR

¹H NMR spectra were recorded on a Bruker AVANCE 400 NMR spectrometer or a Bruker DPX250 NMR spectrometer. Chemical shifts are expressed in parts per million (ppm, δ units). Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

### Purification Techniques

Purification of the Examples may be carried out by conventional methods such as chromatography and/or recrystallisation using suitable solvents. Chromatographic methods include column chromatography, flash chromatography, HPLC (high performance liquid chromatography), SFC (supercritical fluid chromatography), SCX (strong cation exchange chromatography) and MDAP (mass directed autopreparation).

The term “Biotage” when used herein refers to commercially available pre-packed silica gel cartridges.

### Mass Directed Auto Preparation (MDAP)

### Column

Waters Atlantis: 19 mm×100 mm (small scale); and 30 mm×100 mm (large scale). Stationary phase particle size, 5 um.

### Solvents

A: Aqueous solvent=Water+0.1% Formic Acid
B: Organic solvent=Acetonitrile+0.1% Formic Acid
Make up solvent=Methanol: Water 80:20
Needle rinse solvent=Methanol

### Methods

Five methods were used depending on the analytical retention time of the compound of interest:

### (1) Large/Small Scale 1.0-1.5=5-30% B

### (2) Large/Small Scale 1.5-2.2=15-55% B

### (3) Large/Small Scale 2.2-2.9=30-85% B

### (4) Large/Small Scale 2.9-3.6=50-99% B

Runtime, 13.5 minutes, comprising 10-minute gradient followed by a 3.5 minute column flush and re-equilibration step.

### (5) Large/Small Scale 3.6-5.0=80-99% B

Runtime, 13.5 minutes, comprising 6-minute gradient followed by a 7.5 minute column flush and re-equilibration step.

### Flow Rate

20 mls/min (Small Scale) or 40 mls/min (Large Scale).

### Intermediate 1

### (3-Chlorophenyl)methyl 5-{[(3-chlorophenyl)methyl]oxy}-2-methyl benzoate (D1)

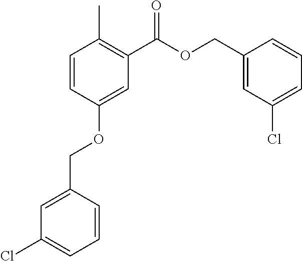

To a solution of 5-hydroxy-2-methylbenzoic acid (1.0 g, 6.6 mmol) in DMF (50 ml) were added potassium carbonate (1.82 g, 13.14 mmol, 2.0 eq) and 3-chlorobenzyl bromide (1.72 g, 13.14 mmol, 2.0 eq). The mixture was stirred at 70° C. for 2 hours and then left stirring at room temperature overnight. The temperature was increased to 80° C. and heating continued for a further 6 hours. After cooling the mixture was diluted with ethyl acetate (200 ml) and washed with water (2×200 ml). Organic layer was separated, dried over MgSO₄, filtered and evaporated to dryness to afford the title compound as a yellow oil, 2.67 g (contains 24% of monoalkylated phenol impurity). No further purification carried out. MS (ES−) m/z 399 [M−H]⁻ (C₂₂H₁₈³⁵Cl₂O₃). ¹H-NMR (400 MHz, CDCl3) δ 2.52 (3H, s), 5.04 (2H, s), 5.29 (2H, s), 7.02 (1H, dd, J 8.4, J 2.8), 7.16 (1H, d, J 8.4), 7.29-7.34 (6H, m), 7.43 (2H, d, J 1.2), 7.54 (1H, d, J 2.8).

### Intermediate 2

### 5-{[(3-Chlorophenyl)methyl]oxy}-2-methyl benzoic acid (D2)

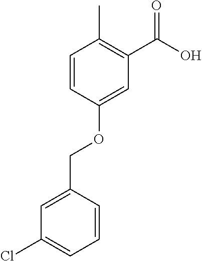

A solution of (3-chlorophenyl)methyl 5-{[(3-chlorophenyl)methyl]oxy}-2-methylbenzoate (D1; 2.67 g, 6.66 mmol) in 1,4-dioxane (20 ml) and water (10 ml) was treated with lithium hydroxide (419 mg, 9.99 mmol, 1.5 eq). The resulting mixture was stirred at room temperature under argon overnight. A further portion of lithium hydroxide (419 mg, 9.99 mmol, 1.5 eq) was added and stirring continued at room temperature for 2 hours. Stirring continued at room temperature overnight. The solvent was then evaporated to dryness and then partitioned between 2M HCl (100 ml) and diethylether (100 ml). The organic layer was separated and passed through a hydrophobic frit to remove any water and evaporated to dryness to afford the title compound as a white solid (1.98 g) (contains 12% impurity). No further purification carried out. MS (ES−) m/z 275 [M−H]⁻ (C₁₅H₁₃³⁵ClO₃).

### Intermediate 3

### 5-{[(3-Chlorophenyl)methyl]oxy}-2-methyl benzoylchloride (D3)

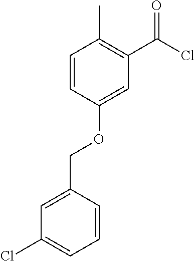

DMF (1 drop) was added to a suspension of 5-{[(3-chlorophenyl)methyl]oxy}-2-methylbenzoic acid (D2; 200 mg, 0.72 mmol) and oxalyl chloride (95 μl, 1.08 mmol, 1.5 eq) in DCM (5 ml). The resulting mixture was stirred at room temperature for 1 hour. The reaction mixture was then evaporated to dryness and azeotroped with toluene (2×50 ml). The organic layer was separated, dried and evaporated in vacuo to afford the title compound as a yellow solid, 213 mgs. No further purification carried out. LCMS sample dissolved in methanol, MS (ES+) m/z 291 [M+H]⁺ (C₁₆H₁₅³⁵ClO₃), corresponding to methyl ester generated from acid chloride.

### Intermediate 4

### Ethyl(4-{[(5-{[(3-chlorophenyl)methyl]oxy}-2-methyl phenyl)carbonyl]amino}-3-methylphenyl)acetate (D4)

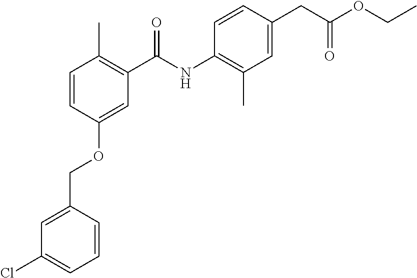

Triethylamine (74 μl, 0.53 mmol, 1.5 eq) was added to a suspension of 5-{[(3-chlorophenyl)methyl]oxy}-2-methylbenzoylchloride (D3; 105 mgs, 0.36 mmol) and ethyl(4-amino-3-methylphenyl)acetate (103 mg, 0.53 mmol, 1.5 eq) in dichloromethane (5 ml). The mixture was stirred at room temperature overnight. The mixture was then diluted with acetonitrile and purified by SCX cartridge (5 g) eluting with acetonitrile. Fractions containing product were combined and evaporated to give the title compound as a yellow gum, 174 mg. MS (ES+) m/z 452 [M+H]⁺ (C₂₆H₂₆³⁵ClNO₄.

### Intermediate 5

### Ethyl 5-hydroxy-2-methylbenzoate (D5)

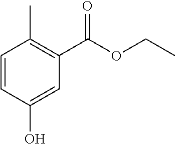

Aluminium chloride (97 g, 731 mmol) was added over 30 seconds to stirred DCM (3 L) under argon at 16° C. resulting in a temp rise to 20° C. When this had dissolved (approx 5 mins) and the temp had cooled to 18° C., ethyl-2-propynate (71.7 g, 731 mmol) was added. A solution of 2-methylfuran (60 g, 731 mmol) in DCM (600 ml) was added to the stirred solution over 35 minutes resulting in a measured exotherm 20.5° C. The exotherm was controlled by a Huber cooling unit and the observed temp range during the addition was 18° C.-20.5° C. After the addition was complete the brown reaction mixture was stirred at 20° C. After a total of 50 mins at 20° C., the reaction mixture was poured into water (3 L) and ice (1 Kg) with stirring to give a yellow mixture. This was transferred to a separating funnel and shaken vigorously.

The layers were separated and the aqueous phase further extracted with DCM (1 L). The combined organic extracts were re-washed with water (1.5 L), dried (Na₂SO₄) and filtered through Kieselguhr. The filtrate was concentrated in vacuo to a brown/green oil. This was purified by silical gel flash chromatography on 2 Biotage 75 L columns in toluene (700 ml) and the solution split into 2 equal portions and each passed through a 75 L column, collecting 400 ml fractions and eluting with the following solvent eluant systems:

### 1st Column:

toluene (3 L)
acetone/toluene (3:97) (2.5 L)
acetone/toluene (6:94) (2.5 L)
acetone/toluene (9:91) (2.5 L)

A moderate separation was achieved. Fraction 14 was recycled into 2nd column separation

Fractions 15-17 combined and contained product

### 2nd Column:

toluene (3 L)
acetone/toluene (1:99) (2.5 L)
acetone/toluene (3:97) (2.5 L)
acetone/toluene (4:96) (2.5 L)
acetone/toluene (5:95) (2.5 L)

Moderate separation achieved. Fraction 17 was mixture and recycled (17 g) into 3rd column separation.

Fraction 18-23 combined and contained product.

### 3rd Column:

The mixture was applied as a solution in toluene (50 ml) to a Biotage 75M column, eluting

as follows and collecting 200 ml fractions.
ethyl acetate/iso-hexane (5:95) (1.5 L)
ethyl acetate/iso-hexane (1:9) (2.5 L)
ethyl acetate/iso-hexane (15:85) (0.2 L)

Reasonable separation achieved.

Fractions 19-28 combined and contained product.

### Pooling of Product Fractions

### F15-17 (C1)

### F18-23 (C2)

### F19-28 (C3)

combined and concentrated in vacuo to a yellow oil which solidified on drying at rt under vacuum for 4 h: Wt=41.7 g, (0.231 mol, 32%). MS (ES⁻) [C₁₀H₁₂O₃—H]⁻ 179. ¹H-NMR (400 MHz) δ 1.38 (3H, t, J 7.2), 2.51 (3H, s), 4.35 (2H, q, J 7.2), 5.36 (1H, s), 6.91 (1H, dd, J 8.4, 2.8), 7.10 (1H, d, J 8.0), 7.43 (1H, d, J 2.8).

### Intermediate 6

### Ethyl 5-{[(3-chlorophenyl)methyl]oxy}-2-methyl benzoate (D6)

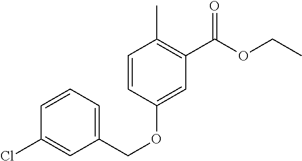

A suspension of ethyl 5-hydroxy-2-methylbenzoate (D5; 39.06 g, 217 mmol), 3-chlorobenzyl bromide (31.3 ml, 238 mmol) and potassium carbonate (44.9 g, 325 mmol) in N,N-dimethylformamide (1000 ml) was stirred at room temperature for 18 hours. The reaction was then filtered, diluted with ethyl acetate (2 L), washed with water (2 L then 3×1 L) and brine (1 L), filtered through a hydrophobic frit and concentrated to give the title compound as a dark yellow oil (68.61 g) which was used without further purification. MS (ES⁺) [C₁₇H₁₇³⁵ClO₃+H]⁺305. ¹H-NMR (400 MHz, d₆-DMSO) δ 1.31 (3H, t, J 7.2), 2.42 (3H, s), 4.28 (2H, q, J 7.2), 5.14 (2H, 2), 7.13-7.16 (1H, m), 7.19-7.23 (1H, m), 7.33-7.45 (4H, m), 7.54-7.55 (1H, m).

### Intermediate 7

### 5-{[(3-Chlorophenyl)methyl]oxy}-2-methyl benzoic acid (D7)

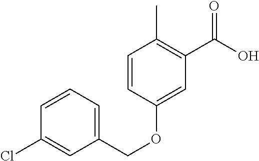

Lithium hydroxide (16.3 g, 389 mmol) was added to a solution of ethyl 5-{[(3-chlorophenyl)methyl]oxy}-2-methylbenzoate (D6; 79 g, 259 mmol) in 1,4-dioxane (1 L) and water (0.5 L). The reaction mixture was stirred at 65° C. for 5 hours, allowed to cool and stood at room temperature for 14 hours. The reaction was concentrated to remove the 1,4-dioxane, and the resulting brown aqueous solution was washed with diethyl ether (3×1 L). The aqueous layer was then acidified with 2N HCl (approximately 200 ml) and the resulting precipitate filtered and washed with water to give a yellow solid. This was dried overnight at 40° C. in a vacuum oven to give the title compound as a yellow solid (66.38 g). MS (ES⁻) [C₁₅H₁₃³⁵ClO₃—H]⁻ 275. ¹H-NMR (400 MHz, d₆-DMSO) δ 2.43 (3H, s), 5.14 (2H, s), 7.10-7.13 (1H, m), 7.21-7.23 (1H, m), 7.38-7.47 (4H, m), 7.52 (1H, s).

### Intermediate 8

### Ethyl phenyl methyl(3-methyl-4-nitrophenyl)propanedioate (D8)

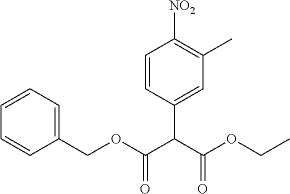

Sodium Hydride (19.09 g, 477 mmol) was added portionwise over 30 minutes to an ice cooled solution of ethyl phenylmethyl propanedioate (D8; 115 ml, 477 mmol) in DMF (500 ml). Upon complete addition, the reaction was allowed to warm to room temperature. After stirring for 30 minutes, a solution of 4-fluoro-2-methyl-1-nitrobenzene (30 ml, 239 mmol) in DMF (250 ml) was added, the reaction heated to 100° C. and stirred at this temperature for 16 hours under an atmosphere of argon. The reaction was then left to stand at room temperature for 72 hours. The reaction was quenched with concentrated HCl (˜100 ml) with stirring and cooling (ice bath), diluted with ethyl acetate (2 L), washed with water (3×2 L), brine (1 L), dried over sodium sulfate, filtered and concentrated to give a dark yellow oil (˜150 g). Purification was by silica chromatography, 1500 g cartridge, sample loaded as a toluene solution to the pre-conditioned column). A gradient of 0 to 5% acetone in pentane eluted the higher running component, this was followed by a gradient of 5 to 15% acetone in isohexane to elute the excess benzyl ethyl malonate and required product. Product-containing fractions were concentrated to give the title compound as a pale yellow oil (71.21 g). MS (ES⁻) [C₁₉H₁₉NO₆—H]⁻ 356. ¹H-NMR (400 MHz, d₆-DMSO) δ 1.14 (3H, t, J 7), 2.50 (3H, s), 4.01-4.21 (2H, m), 5.14-5.25 (2H, m), 5.27 (1H, s), 7.28-7.49 (7H, m), 8.00 (1H, d, J 8)

### Intermediate 9

### Ethyl(4-amino-3-methylphenyl)acetate (D9)

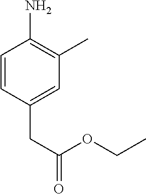

Batch 1: Ethyl phenylmethyl(3-methyl-4-nitrophenyl)propanedioate (35 g, 98 mmol) was taken up in Ethanol (500 ml) and subjected to a hydrogenation at atmospheric pressure using 10% palladium on carbon (3.13 g, 2.94 mmol). After 18 hours a small sample was removed for analysis. The catalyst was removed by filtration through celite and fresh catalyst palladium on carbon (3.13 g, 2.94 mmol) was added to the reaction mixture. The reaction was put on again for 3 hours. The hydrogenation continued for a further 3 hours. The hydrogenation reservoir was refilled and the reaction continued overnight. The catalyst was removed by filtration through celite and fresh catalyst palladium on carbon (3.13 g, 2.94 mmol) was added to the reaction mixture. The reaction was put on again for 3 hours. After the weekend stirring under these conditions, the reaction mixture added to the equivalent reaction mixture from Batch 2.
Batch 2: ethyl phenylmethyl(3-methyl-4-nitrophenyl)propanedioate (35 g, 98 mmol) was taken up in Ethanol (500 ml) and subjected to a hydrogenation at atmospheric pressure using 10% palladium on carbon (3.13 g, 2.94 mmol). After 2 hours a sample was taken for analysis. The reservoir was refilled with hydrogen and the reaction continued overnight. The catalyst was removed by filtration through celite and fresh catalyst palladium on carbon (3.13 g, 2.94 mmol) was added to the reaction mixture. The reaction was put on
again for 3 hours. After leaving the reaction mixture under these conditions for the weekend, it was combined with BATCH 1 reaction mixture (equivalent reaction mixture) and filtered through celite to give a colourless filtrate. This solution was concentrated to an oily solid (wt=31.9 g). To this mixture was added toluene (200 ml) and the mixture was filtered from an insoluble white gum (wt=0.65 g). The filtrate was passed down a silica gel Biotage 75 L chromatography column eluting with the following ethyl acetate/iso-hexane gradient mixture and collecting 400 ml fractions:
ethyl acetate/iso-hexane
440 ml:2500 ml (15%)
833:2500 ml (25%)
1000:1900 ml (35%)
800:1250 ml (40%)

Fractions 13-19 were combined and concentrated to give the title compound as an oil which solidified on drying for 2 hours at room temperature under vacuum (wt=24.5 g). MS (ES⁺) [C₁₁H₁₅NO₂+H]⁺194. ¹H-NMR (400 MHz, d₆-CDCl₃) δ 1.25 (3H, t, J 7), 2.15 (3H, s), 3.48 (s, 2H), 3.56 (2H, br. s), 4.13 (2H, q, J 7), 6.63 (1H, d, J 8), 6.93-6.97 (2H, m).

### Intermediate 10

### Ethyl(4-{[(5-{[(3-chlorophenyl)methyl]oxy}-2-methylphenyl)carbonyl]amino}-3-methylphenyl)acetate (D10)

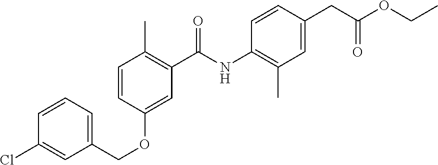

Oxalyl chloride (15.1 ml, 173 mmol) was added over approx 1 minute to a stirred suspension of 5-{[(3-chlorophenyl)methyl]oxy}-2-methylbenzoic acid (D7; 31.8 g, 115 mmol) in dichloromethane (1.14 L) at 20° C. under argon. This was followed by the addition of N,N dimethylformamide (2 ml, 25.8 mmol) over 3 minutes with accompanying gas evolution but no noticeable temperature rise. Within approx 15 minutes the suspension dissolved and turned a darker brown. The mixture was stirred under argon at 20° C. for a total of 75 minutes. After 75 minutes the reaction mixture was evaporated in vacuo to a cream solid which was dried under vacuum at room temperature for 1 h. The solid was then redissolved in dichloromethane (900 ml) and to the stirred solution at 20° C. under argon was simultaneously added a solution of ethyl(4-amino-3-methylphenyl)acetate (D9; 24.4 g, 126 mmol) in dichloromethane (240 ml) and triethylamine (24 ml) over 10-15 minutes. The temperature rise in the reaction was from 20° C. to 30° C. over this time and thereafter slowly cooled to ambient. The brown solution was stirred under argon at room temperature for 14 h. After 14 h stirring the brown solution was washed with water (2×1 L). The combined aqueous washes were re-extracted with dichloromethane (500 ml) and all the organic extracts were combined, dried (MgSO₄) and evaporated in vacuo to a brown oily solid which was dried at room temperature, under vacuum for 0.5 hours (wt=51.7 g). This material was dissolved in dichloromethane (200 ml) and the solution column chromatographed on Biotage 75 L system eluting with an ethyl acetate/iso-hexane gradient mixture and collecting 400 ml fractions as follows:

ethyl acetate/iso-hexane 440 ml/2500 ml (15%)
833 ml/2500 ml (25%)
2016 ml/3750 ml (35%)

Fractions 15-20 were combined and concentrated to a pink solid, dried at room temperature under vacuum for 2 hours (Wt=43.2 g). The solid was stirred with diethyl ether (100 ml) for 1 h at room temperature to remove most of the colour in the supernatant. The off-white solid was filtered and dried at 40° C. under vacuum for 4 hours. Wt=40.8 g. MS (ES⁺) [C₂₆H₂₆³⁵ClNO₄⁺H]⁺452. ¹H-NMR (400 MHz) δ 1.26 (3H, t, J 7.2), 2.26 (3H, s), 2.45 (3H, s), 3.57 (2H, s), 4.15 (2H, q, J 7.2), 5.06 (2H, s), 6.95-7.33 (10H, m), 7.94 (1H, d, J 8)

### EXAMPLE 1

### Preparation of (4-{[(5-{[(3-chlorophenyl)methyl]oxy}-2-methylphenyl)carbonyl]amino}-3-methylphenyl)acetic acid

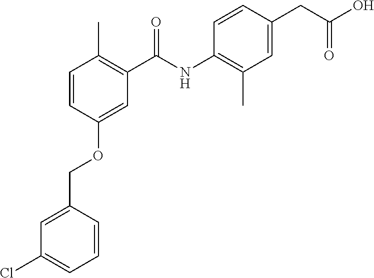

Ethyl(4-{[(5-{[(3-chlorophenyl)methyl]oxy}-2-methylphenyl)carbonyl]amino}-3-methylphenyl)acetate from the preparation of Intermediate 4 described above (174 mg, 0.39 mmol) was taken up in acetic acid (10 ml) and 2M HCl (10 ml) and heated at 90° C. for 2 hours. The reaction mixture was allowed to cool and stirring continued at room temperature overnight. The reaction mixture was extracted with ethyl acetate (50 mls), dried using a hydrophobic frit and then evaporated to dryness to give a yellow solid/gum, 155 mg. This was purified by MDAP to give the title compound as a white solid, 39 mg. MS (ES+) m/z 424 [M+H]⁺ (C₂₄H₂₂³⁵ClNO₄). ¹H-NMR (400 MHz, d₆-DMSO) δ 2.24 (3H, s), 2.34 (3H, s), 3.52 (2H, s), 5.17 (2H, s), 7.02-7.15 (4H, m), 7.22 (1H, d, J 8.4), 7.31 (1H, d, J 8.0), 7.39-7.44 (3H, m), 7.53 (1H, s), 9.71 (1H, s), 12.33 (1H, bs).

### EXAMPLE 2

### Large Scale Preparation of (4-{[(5-{[(3-chlorophenyl)methyl]oxy}-2-methylphenyl)carbonyl]amino}-3-methylphenyl)acetic acid

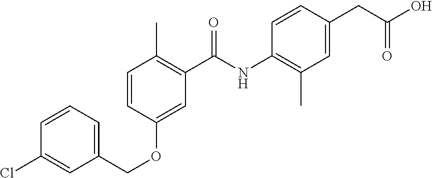

To a stirred solution of ethyl(4-{[(5-{[(3-chlorophenyl)methyl]oxy}-2-methylphenyl)carbonyl]amino}-3-methylphenyl)acetate (D10; 39.8 g, 88 mmol) in 1,4-dioxane (700 ml) at ambient temp. under argon was added a solution of lithium hydroxide monohydrate (5.5 g, 131 mmol) in water (400 ml) and the stirred pink solution was then heated to a block temperature of 65° C. The following internal temps were noted during the course of the reaction:

t=0 mins 20° C.
t=40 mins 47° C.
t=60 mins 50° C.
t=75 mins 50° C.

After 75 mins the reaction mixture was cooled to 40° C. and concentrated in vacuo (bath temp 40° C.) to remove 650 ml of solvent, at which time crystallisation began to occur in the reaction mixture. At this point 2M hydrochloric acid (150 ml) was added to the mixture causing further crystallisation and turning the colour from pink to yellow. The supernatant was measured as pH1 and the mixture was further concentrated to remove another 80 ml of solvent. The stirred mixture was then cooled to 5° C. in an ice bath and after 0.5 h it was filtered off and washed with water (3×150 ml), sucked dry then further dried at 40° C., under vacuum, for 20 hours (wt=37.4 g). MS (ES⁺) [C₂₄H₂₂³⁵ClNO₄+H]⁺424. ¹H-NMR (400 MHz, d₆-DMSO) δ 2.24 (3H, s), 2.35 (3H, s), 3.53 (2H, s), 5.17 (2H, s), 7.02-7.53 (10H, m), 9.72 (1H, s), 12.35 (1H, br. s).

### EXAMPLE 3

### Preparation of (4-{[(5-{[(3-chlorophenyl)methyl]oxy}-2-methylphenyl)carbonyl]amino}-3-methylphenyl)acetate sodium salt

2 mL of MeOH was added to 100 mg of partially crystalline (4-{[(5-{[(3-chlorophenyl)methyl]oxy}-2-methylphenyl)carbonyl]amino}-3-methylphenyl)acetic acid and the slurry was heated with a hot air gun until complete dissolution. 23.5 μL of 10M sodium hydroxide solution was added to 0.5 mL of MeOH and sonicated to ensure thorough mixing. The counter-ion solution was then added drop-wise to the drug substance solution and the mixture was left stirring gently at room temperature overnight.

After ˜12 hours, the solution had formed a birefringent slurry (confirmed by PLM analysis) and was filtered under vacuum. The solid was then dried at 40° C. in vacuo overnight.

### EXAMPLE 4

### Preparation of (4-{[(5-{[(3-chlorophenyl)methyl]oxy}-2-methylphenyl)carbonyl]amino}-3-methylphenyl)acetate potassium salt

1.5 mL of MeOH was added to 50 mg of partially crystalline (4-{[(5-{[(3-chlorophenyl)methyl]oxy}-2-methylphenyl)carbonyl]amino}-3-methylphenyl)acetic acid and the slurry was heated with a hot air gun until complete dissolution. 10 μL of potassium hydroxide solution (45% solution in water) was added to the drug substance solution and the mixture was left stirring gently at room temperature overnight.

After ˜12 hours, no precipitation was evident therefore ˜0.5 mL of MeOH was evaporated under nitrogen and the glass vial was scratched with a spatula to encourage nucleation. Particles were observed in solution, therefore the mixture was left to crystallise with continuous stirring over the weekend.

After stirring over the weekend, the solution had formed a birefringent slurry (confirmed by PLM analysis) and was filtered under vacuum. The solid was then dried at 40° C. in vacuo overnight.

### EXAMPLE 5

### Preparation of (4-{[(5-{[(3-chlorophenyl)methyl]oxy}-2-methylphenyl)carbonyl]amino}-3-methylphenyl)acetate methyl-D-glucamine salt

2 mL of MeOH was added to 50 mg of partially crystalline (4-{[(5-{[(3-chlorophenyl)methyl]oxy}-2-methylphenyl)carbonyl]amino}-3-methylphenyl)acetic acid and the slurry was heated with a hot air gun until complete dissolution. 23.4 mg of N-methyl-D-glucamine was dissolved in 1 mL of MeOH using a hot air gun. The counter-ion solution was then added drop-wise to the drug substance solution. The mixture was left stirring gently at room temperature overnight.

After ˜12 hours, no precipitation was evident therefore ˜1 mL of MeOH was evaporated under nitrogen. The mixture was then placed in an ice bath for ˜2 hours to promote crystallisation. As no precipitation was evident, the remaining solvent was evaporated leaving a gum.

The gum was dissolved up in 2 mL of EtOAc with sonication, leaving a thick slurry. The slurry was left stirring gently at room temperature over the weekend in an attempt to improve the birefringence.

After the stirring over the weekend, a further 1 mL of EtOAc was added to the slurry and sonicated, to thin the slurry down for filtering. It was then filtered under vacuum and dried at 40° C. in vacuo overnight.

### EXAMPLE 6

### Preparation of (4-{[(5-{[(3-chlorophenyl)methyl]oxy}-2-methylphenyl)carbonyl]amino}-3-methylphenyl)acetate tris(hydroxymethyl)-aminomethane salt

2 mL of acetone was added to 100 mg of partially crystalline (4-{[(5-{[(3-chlorophenyl)methyl]oxy}-2-methylphenyl)carbonyl]amino}-3-methylphenyl)acetic acid and the slurry was heated with a hot air gun until complete dissolution. 2 mL of acetone was added to 28.8 mg of tris(hydroxymethyl)-aminomethane and the mixture was heated and sonicated but did not dissolve. Therefore, the drug substance solution was added drop-wise to the counter-ion solution and the mixture was left stirring gently at room temperature. Immediate precipitation was observed. Stirring was continued overnight.

After ˜12 hours, the birefringent slurry (confirmed by PLM analysis) was filtered under vacuum and dried at 40° C. in vacuo overnight.

1. A compound which is (4-{[(5-{[(3-chlorophenyl)methyl]oxy}-2-methylphenyl)carbonyl]amino}-3-methylphenyl)acetate sodium salt.

2. A compound which is (4-{[(5-{[(3-chlorophenyl)methyl]oxy}-2-methylphenyl)carbonyl]amino}-3-methylphenyl)acetate potassium salt.

3. A compound which is (4-{[(5-{[(3-chlorophenyl)methyl]oxy}-2-methylphenyl)carbonyl]amino}-3-methylphenyl)acetate methyl-D-glucamine salt.

4. A compound which is (4-{[(5-{[(3-chlorophenyl)methyl]oxy}-2-methylphenyl)carbonyl]amino}-3-methylphenyl)acetate tris(hydroxymethyl)-aminomethane salt.

5. A pharmaceutical composition comprising a compound of any one of claims 1 to 4 and a pharmaceutically acceptable carrier or diluent thereof.

6. A compound according to any one of claims 1 to 4 for use in the treatment of a condition which is mediated by the action, or loss of action of PGE₂ at EP₄ receptors.

7. A compound according to any one of claims 1 to 4 for use in the treatment of a Bone Disorder.

8. A method of treating a human or animal subject suffering from a condition which is mediated by the action, or by loss of action, of PGE₂ at EP₄ receptors which comprises administering to said subject an effective amount of a compound according to any one of claims 1 to 4.

9. A method of treating a human or animal subject suffering from a Bone Disorder which comprises administering to said subject an effective amount of a compound according to any one of claims 1 to 4.

10. Use of a compound of any one of claims 1 to 4 for the manufacture of a medicament for the treatment of a condition which is mediated by the action, or by loss of action, of PGE₂ at EP₄ receptors.

11. Use of a compound of any one of claims 1 to 4 for the manufacture of a medicament for the treatment of a Bone Disorder.

12. A pharmaceutical composition comprising a compound of any one of claims 1 to 4 and a pharmaceutically acceptable carrier or diluent thereof.

13. A pharmaceutical composition according to claim 12 comprising one or more additional therapeutic agents.

14. Compound according to claim 6 or method according to claim 8 or use according to claim 10 wherein the condition is pain.

15. Compound or method or use according to claim 14 wherein the pain condition is selected from the group consisting of chronic articular pain; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections; rheumatic fever; pain associated with functional bowel disorders; pain associated with myocardial ischemia; post operative pain; headache; toothache and dysmenorrhea.

16. Compound according to claim 7 or method according to claim 9 or use according to claim 11 wherein the Bone Disorder is selected from the group consisting of fracture healing, bone grafting, a periodontal indication and malignant bone tumour e.g. osteosarcoma.
